(19) United States
(12) Patent Application Publication
(10) Pub. No.: US 2023/0126877 A1
(43) Pub. Date: Apr. 27, 2023

(54) SYNTHETIC DATA GENERATION AND ANNOTATION FOR TUMOR DIAGNOSTICS AND TREATMENT

(71) Applicant: DYNAM.AI, Inc., San Diego, CA (US)

(72) Inventors: Dimitry FISHER, San Diego, CA (US); Diana SHAPIRO, I, San Diego, CA (US)

(21) Appl. No.: 17/973,446

(22) Filed: Oct. 25, 2022

Related U.S. Application Data
(60) Provisional application No. 63/360,754, filed on Oct. 25, 2021.

(51) Int. Cl. G06V 10/774; G06T 7/00; G16H 50/50

(52) U.S. Cl. CPC G06V 10/774; G06T 7/0014; G16H 50/50; G06T 2207/30096; G06T 2207/20081

(57) ABSTRACT

Certain aspects of the present disclosure provide techniques for training a tumor detection model. A method generally includes processing an input scan image of a tumor in a first state using a first computational model configured to simulate growth of the tumor in a first configuration to generate model output images of the simulated tumor comprising at least a first model output image representing the tumor in a second state, wherein the first model output image comprises a timestamp associated with the second state, training a first machine learning model to convert the first model output image to a first synthesized scan image, and training a second machine learning model to detect the simulated tumor in image data using the first synthesized scan image and the input scan image.

### CROSS-REFERENCE TO RELATED APPLICATIONS

This Application claims the benefit of and priority to U.S. Provisional Patent Application Ser. No. 63/360,754, entitled “Apparatus and methods for tumor diagnostics and therapy,” filed Oct. 25, 2021, the contents of which are hereby incorporated by reference in their entirety.

### INTRODUCTION

Aspects of the present disclosure relate to machine learning, and in particular to training and using machine learning models for synthetic data generation, annotation, and prediction.

### BACKGROUND

Cancer is one of the leading causes of death in the world. Cancer is a disease in which some of the body's cells grow uncontrollably and spread to other parts of the body. Generally, human cells grow and multiply (e.g., through a process commonly referred to as “cell division”) to form new cells as the body needs them. When cells grow old or become damaged, they die, and new cells take their place. Sometimes this orderly process breaks down, and abnormal or damaged cells grow and multiply when they should not. These cells may form tumors, which are lumps of tissue. Tumors can be malignant (e.g., cancerous) or benign (e.g., not cancerous). Cancerous tumors spread into, or invade, nearby tissues and can travel to distant places in the body to form new tumors (e.g., a process commonly referred to as “metastasis”).

Detecting and diagnosing cancer at its earliest stages often provides the best chance for effective treatment and patient survival. Traditionally, physicians use one or more approaches to diagnose cancer in a patient, including but not limited to, conducting a physical exam, performing one or more laboratory tests including urine and/or blood tests, performing one or more imaging tests including, for example, a computerized tomography (CT) scan, a bone scan, a magnetic resonance imaging (Mill) scan, a functional MRI (fMRI) scan, a positron emission tomography (PET) scan, an ultrasound, and/or an X-ray, performing a biopsy to remove a piece of tissue and/or a sample of cells from the body for testing in a laboratory, and/or the like. Additionally, the physician may determine one or more treatment options (e.g., chemotherapy, immunotherapy, surgery, electromagnetic radiation, particle beams, etc.) for the patient based on results of performing one or more of the above-described diagnostics tests. As such, diagnostics and treatment of cancer has traditionally been the responsibility of a physician (and/or radiologist), with little or no help from artificial intelligence (AI). Unfortunately, it has been common for different human diagnosticians to render different diagnoses based on the same underlying data, which is generally undesirable, and which can significantly impact the patient outcome.

AI is an umbrella term describing the mimicking of human intelligence by computers. Machine learning, a subdivision of AI, refers to training computer algorithms to make predictions based on experience, and can be broadly divided into supervised (e.g., where a computer is allowed to see the outcome data) or unsupervised (e.g., where no outcome data is provided) learning. Both approaches look for data patterns to allow outcome predictions, such as the presence or absence of cancer/tumors. As such, AI and machine learning has the potential to revolutionize cancer diagnostics and treatment decisions; however, deployment and use of these techniques is limited due to the lack of available (and/or quality of) clinical data.

Indeed, modern AI approaches are dependent on the availability of large, high-quality datasets. In general, the more facets the data covers, the faster the algorithms can learn and fine-tune their output (e.g., an inference of whether cancer exists or not in a scan image). In some cases, if the data used for training machine learning models is not sufficiently diverse and/or unbiased, problems such as artificial “AI bias” may arise. AI bias is an anomaly in which the inherent bias of training data causes a model trained based on that data to inherent the same bias in function. So, for example, a homogeneous data set used to train a model may generate a model that does not predict well with respect to a heterogeneous data set.

It has been well recognized in the past decade, that an obstacle to effectively using machine learning for tumor diagnostics and treatment is related to the insufficient amount of quantitative clinical data, such as X-rays, CT scans, PET scans, MRI scan, and/or fMRI scans, available for specific pathologies (e.g. different tumor types, infectious diseases, and/or autoimmune diseases). Additionally, lack of context, such as human-made annotations (e.g., provided by physicians), including identifying tumor areas and/or other pertinent information, in these scans further contributes to the minimal use of AI and/or machine learning techniques for cancer/tumor diagnosis and treatment. For example, the lack of available and quality clinical data hinders the ability of conventional machine learning approaches to learn correct diagnostic features for use in predictive analyses.

Accordingly, improved techniques for synthetic data generation, which may be used in training machine learning models for tumor diagnostics and/or treatment, are desired.

### SUMMARY

Certain embodiments provide a method for training a tumor detection model. The method generally includes processing an input scan image of a tumor in a first state using a first computational model configured to simulate growth of the tumor in a first configuration to generate model output images of the simulated tumor comprising at least a first model output image representing the tumor in a second state, wherein the first model output image comprises a timestamp associated with the second state, training a first machine learning model to convert the first model output image to a first synthesized scan image, and training a second machine learning model to detect the simulated tumor in image data using the first synthesized scan image and the input scan image.

Certain embodiments provide a method for generating synthesized scan images representing a tumor. The method generally includes processing an input scan image of the tumor in a first state using a first computational model configured to simulate growth of the tumor in a first configuration to generate model output images of the simulated tumor comprising at least a first model output image representing the tumor in a second state, wherein the first model output image comprises a timestamp associated with the second state, converting the first model output image to a first synthesized scan image using a first machine learning model trained to convert model output images to synthesized scans, and detecting the simulated tumor in the first synthesized scan image using a second machine learning model trained to simulated tumors in image data.

Certain embodiments provide another method for generating synthesized scan images representing a tumor. The method generally includes generating predicted model output images of a tumor simulation for the tumor at a predetermined time step for a period of time, wherein a plurality of predicted model output images of the simulated tumor are generated and represent different states of the tumor for the period of time, converting the plurality of predicted model output images to a plurality of synthesized scan images using a first machine learning model trained to convert predicted model output images to synthesized scans, and performing at least one of: detecting the simulated tumor in each of the plurality of synthesized scan images using a second machine learning model trained to simulated tumors in image data or generating one or more synthesized scan images representing the tumor in a state in time between at least two of the plurality of predicted model output images.

Other embodiments provide processing systems configured to perform the aforementioned methods as well as those described herein; non-transitory, computer-readable media comprising instructions that, when executed by one or more processors of a processing system, cause the processing system to perform the aforementioned methods as well as those described herein; a computer program product embodied on a computer readable storage medium comprising code for performing the aforementioned methods as well as those further described herein; and a processing system comprising means for performing the aforementioned methods as well as those further described herein.

The following description and the related drawings set forth in detail certain illustrative features of one or more embodiments.

### DESCRIPTION OF THE DRAWINGS

The appended figures depict certain aspects of the one or more embodiments and are therefore not to be considered limiting of the scope of this disclosure.

FIG. 1A illustrates an example system for synthetic data generation and tumor detection, according to aspects of the present disclosure.

FIG. 1B illustrates an example model output image compared to an example synthetic scan image, according to aspects of the present disclosure.

FIG. 2A illustrates example operations for training a tumor detection model, according to aspects of the present disclosure.

FIG. 2B illustrates example operations for training a machine learning model to convert computational model output to synthesized scan image(s), according to aspects of the present disclosure.

FIG. 2C illustrates example operations for training a machine learning model to detect a tumor in a synthesized scan image, according to aspects of the present disclosure.

FIG. 3 illustrates another example system for synthetic data generation and tumor detection, according to aspects of the present disclosure.

FIG. 4A illustrates example operations for training another tumor detection model, according to aspects of the present disclosure.

FIG. 4B illustrates example operations for training a machine learning model to generate predicted model output image(s) representing different states of a simulated tumor over a period of time, according to aspects of the present disclosure.

FIG. 4C illustrates example operations for training a machine learning model to generate synthesized scan image(s) representing a future and/or past state of a tumor, according to aspects of the present disclosure.

FIG. 5 illustrates an example system on which aspects of the present disclosure can be performed.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Data augmentation is a method that is used to artificially increase the amount of available data by making slightly modified copies of already existing data or newly created synthetic data from existing data. In some cases, this can be as simple as making small changes to datasets to generate artificial data points. For example, data augmentation techniques are used to generate synthetic data (e.g., synthetic scans) by evaluating first and second order statistics of human-annotated tumor areas identified in historical scans for patients with a history of cancerous tumors. As such, a larger synthetic dataset may be formed from which a machine learning model trained to aid in tumor diagnostics and/or treatment can learn from, in addition to the smaller clinical dataset (e.g., annotated scans of tumors for historical patients). Data augmentation may help to improve the predictive accuracy of such models; however, there are limits to using such techniques. First, in some cases, available clinical data may be too limited (e.g., for a particular pathology); thus, data augmentation may not be feasible. Second, in some cases, data augmentation may result in a biased synthetic data set. For example, where available clinical data contains biases, the data augmented from this limited data set will also contain biases. Biased data sets may lead to skewed outcomes, systematic prejudice, and/or low accuracy when used to train machine learning models.

Accordingly, aspects of the present disclosure provide apparatuses, methods, processing systems, and computer-readable mediums for training machine learning models to generate synthetic scan images of a tumor having the tumor detected, which can be in-turn used to train improved diagnostic models (e.g., tumor detection models). For example, one or more computational models and/or machine learning models may be used in combination to generate synthetic representations of past, present, and/or future predicted states of the tumor. The synthetic scan images generated by the models may include computerized tomography (CT) scans, magnetic resonance imaging (MM) scans, functional MRI (fMRI) scans, positron emission tomography (PET) scans, three-dimensional (3D) models, and/or the like. The synthetic scan images may additionally include annotated margins, generated by one or more of the machine learning models, identifying the tumor, as well as a predicted location of the tumor, in the synthetic image data. The annotated tumor margins may provide insight into growth of the tumor, where multiple synthetic scan images are generated to represent different states of the tumor over a period of time. This information may be useful to guide invasive and/or noninvasive treatment for a patient, and in some cases, may be used to quantify the effect of treatment for an individual patient or a cohort of patients such that the treatment may be adjusted accordingly.

In certain aspects, the models used to generate the annotated, synthetic scan images include a computational model, an imaging module, and a plurality of machine learning models (e.g., in some embodiments, referred to herein as an “image adaptation model” and a “predictor model”). For example, the computational model is used to simulate and model tumor growth using mathematics, physics, and computer science. The simulated tumor may represent an actual tumor (e.g., of a current or historical patient), a tumor growing in vitro, and/or a tumor growing in vivo, for example in a laboratory animal. The imaging module, or other similar component, may capture model output images of the simulated tumor at different times during the simulation. The imaging module may be configured to capture images of the simulated tumor continuously and/or frequently (e.g., with an insignificant time step) such that the model output images captured adequately represent a lifecycle of the simulated tumor (e.g., model output images representing past, present, and future states of the simulated tumor). The captured images, and in some cases, metadata for the images, may be used as input into a first machine learning model to produce synthesized scan images representing different states of the tumor over the lifecycle of the tumor. Further, a second machine learning model may be used to detect the simulated tumor in each of the synthesized scan images, and in some cases, further annotate a margin of the simulated tumor in the synthetic scan image. The first machine learning model and the second machine learning model may be trained prior to making such predictions.

In certain other aspects, the models used to generate the annotated, synthetic scan images include an image predictor model and a plurality of machine learning models (e.g., in some embodiments, referred to herein as an “image adaptation model” and a “predictor model”). For example, instead of using a computational model to simulate tumor growth such that model output images may be obtained, the image predictor model may be trained to predict model output images based on an input scan image of a tumor. The input scan image may be an actual tumor (e.g., of a current or historical patient), a tumor growing in vitro, and/or a tumor growing in vivo, for example in a laboratory animal. The predicted model output images generated by the model may a time-ordered sequence of model output images spaced apart in time by a defined time step. For example, where the image predictor model generates three model output images, a first model output image may represent the state of the tumor one month prior, the second model output image may represent the state of the tumor currently, and the third model output image may represent a predicated state of the tumor one month in the future. The predicted model output images, and in some cases, metadata for the images, may be used as input into a first machine learning model trained to convert the predicted model output images to synthesized scan images. A second machine learning model may be used to generate, from the synthesized scan images, additional synthesized scan images representing the tumor in a state in time between at least two of the previously generated synthesized scan images. In other words, the second machine learning mode may be trained to perform interpolation to generate one or more synthesized scan images. For example, where the first machine learning model produces synthesized scan images representing the state of the tumor currently and a predicated state of the tumor one month (e.g., 30 days) in the future, the second machine learning model may be used to generate one or more synthesized scans representing the state of the tumor between today and one month in the future (e.g., a synthesized scan 15 days in the future). Additionally, in some cases, the second machine learning model may be used to detect the tumor in each of the synthesized scan images, and in some cases, further annotate a margin of the simulated tumor in the synthetic scan image. The first machine learning model and the second machine learning model may be trained prior to making such predictions.

Use of the image predictor model in place of the computational model may be based on a compute budget and/or power budget of a system where the models are implemented. In particular, computational modeling, as described herein, may demand a large amount of resources and/or power to simulate the growth of a tumor. Additionally, as more tumors and/or tumor configurations are needed to be modeled to generate model output images for additional synthetic data generation, this demand may significantly increase. Excess resources and/or power allocated to the computational model for processing may have an adverse effect on the overall performance of the system. As such, in some cases, an image predictor model may be used in place of the computational model. In particular, the image predictor model may be trained to generate model output images of a tumor should the tumor be simulated, without actually having to simulate the tumor using a computational model. As such, compute resources and/or power may be saved, thus representing a technical solution and improvement to the technical problem posed by existing methods that rely on heavyweight computational models alone.

Additionally, the first machine learning model may output its internal or latent state, or a representation thereof. These may be provided as an input, or as an additional input, to the second machine learning model or to other components of the present invention, for example to improve their learning or performance. Towards that end, in certain aspects, the first machine learning model, or parts thereof, may function as a feature extractor and encoder.

In certain aspects, the synthetic scan images generated using the techniques described above may help to overcome some of the key problems of using machine learning models in medicine, for example, the limited amount of quantitative data available for training such machine learning models. In particular, the synthetic scan images generated using the techniques described above help to increase the pool of available training data (e.g., quantitative clinical data such as X-rays, CT scans, PET scans, MRI scan, and/or fMRI scans, available for specific pathologies) needed to train machine learning models in medicine, for example, models used to aid in tumor diagnostics and/or treatment decisions. This additional, quality data may cover more facets thereby allowing machine learning algorithms to learn and fine-tune their predictive analyses, while also helping to avoid bias of the machine learning model itself. Accordingly, the methods described herein represent a technical solution and improvement to the technical problem and limitation posed by existing machine learning techniques in which insufficient data is available to train reliable, and generalizable, models.

Additionally, the synthetic scan images generated using the techniques described above help to overcome the inability of present-day medical machine learning models to effectively use context and/or information not contained in the training data to make predictions. In particular, the synthetic scan images generated herein may be generated with one or more annotations, such as annotated tumor margins identifying a detected tumor and its location, area, boundaries, contours, center, etc. These annotations may be context generated from a computational model, as opposed to manually created using data augmentation techniques. As such, these annotations may provide useful and enhanced input to machine learning models trained to aid in tumor diagnostics and/or treatment decisions (e.g., such as surgery and/or radiotherapy decisions). Accordingly, the methods described herein provide a technical solution and improvement to the technical problem of existing machine learning techniques that are not able to generate useful annotations and other metadata that is beneficial to diagnostic accuracy and success.

Additionally, the techniques described herein do not merely augment data and/or overcome data biases. Instead, by combining input data (e.g., input scan images with or without annotations) with computational model outputs and/or with expert feedback, the techniques described herein help to not only augment, but also enrich the data, by adding information not contained and/or not salient in the original data set(s).

Further, some of the synthetic scan images generated herein may provide information about early stages of a tumor, which is information that is not abundantly available in the field of medicine. In particular, very little, or almost no, clinical data (e.g., scans) exists for tumors at their earliest stages. Although high-tech imaging, tissue sample collection, and/or surgical exploration is often used to try and detect tumors early on, these techniques are not always successful. Thus, by the time a tumor is detected and clinical scans are taken, the tumor may already be in its later stages. As such, the techniques herein help to alleviate the shortage of clinically available data with respect to the beginning stages of a tumor's life. Thus, training data used to train machine learning models for tumor diagnostics and treatment may be more diverse and complete, given the addition of this data which may not have been previously present. Accordingly, the methods described herein provide a technical solution and improvement to the technical problem of existing machine learning techniques that are limited by insufficient training data for early stage diagnostics.

### Example Synthetic Data Generation and Annotation using a Computational Model

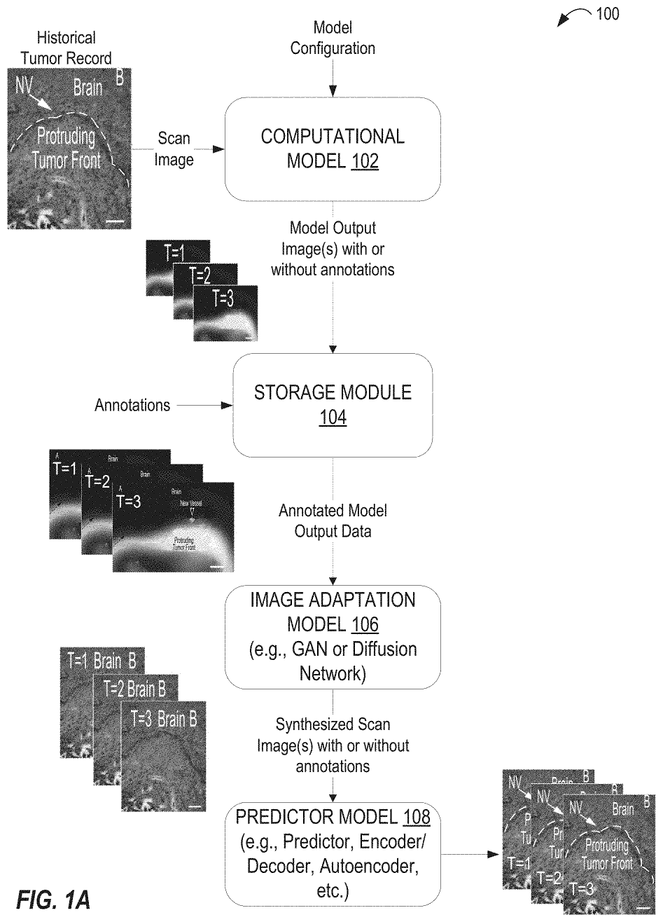

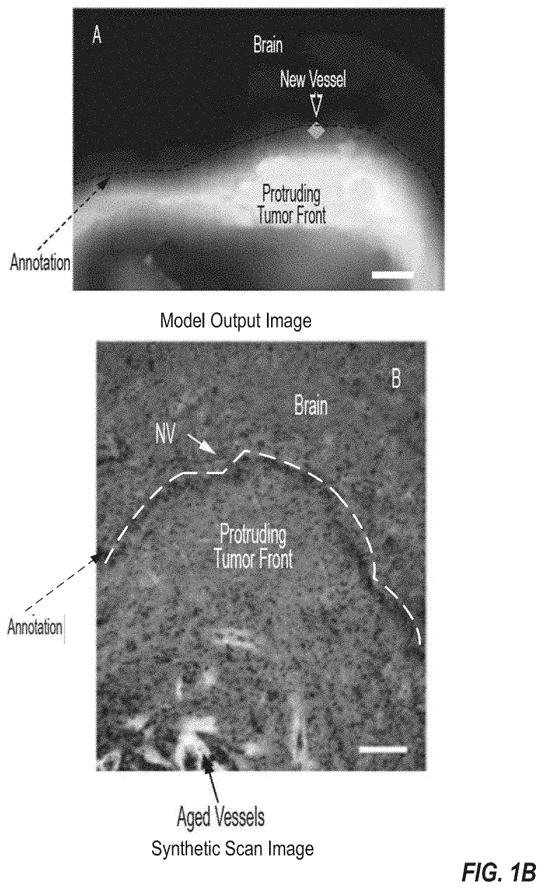

FIG. 1A illustrates an example system 100 for synthetic data generation and tumor detection, according to certain aspects of the present disclosure. Example system 100 includes a computational model 102, a storage module 104, an image adaptation model 106, and a predictor model 108.

Computational model 102 is a mathematical model configured to simulate tumor growth for a specific tumor type (e.g., glioblastoma) and/or a plurality of tumor types. Computational model 102 may model tumor progression through multiple stages including tumor initiation, avascular growth, vascular growth, and/or metastasis. In particular, it has been well established that solid tumors grow in two distinct phases: the initial growth being referred to as the avascular phase and the later growth as the vascular phase. The transition of solid tumor growth from the relatively harmless avascular phase to the invasive and malignant vascular phase depends upon the process of angiogenesis. Tumor angiogenesis refers to the proliferation of a network of blood vessels which supplies a tumor with a supportive microenvironment rich with oxygen and nutrients to sustain optimal growth. During the metastatic phase, the tumor (e.g., cancer cells) may spread to other parts of the body. Computational model 102 may model one or more of the stages to provide a complete representation of the lifecycle of a tumor being simulated via use of computational model 102. In some cases, computation model 102 is representative of one or more underlying models, e.g., a plurality of computational models that may generate the necessary model output.

As illustrated in FIG. 1A, computational model 102 may simulate tumor growth by processing one or more input scan images of a tumor of a current or historical patient, a tumor growing in vitro, and/or a tumor growing in vivo, for example in a laboratory animal. The input scan image(s) used as input into computational model 102 may include X-rays, CT scans, PET scans, MRI scans, and/or fMRI scans for a particular pathology. In certain aspects, the input scan image(s) include one or more medical image annotations provided by clinical experts. The medical image annotations may include, for example, identification of whether a tumor exists in the image data, a location of the tumor, a size of the tumor, an indication of how the tumor has affected nearby tissue, etc. These annotations may be annotations that are understood by computational model 102.

In certain aspects, the input scan image may be a synthetic, artificial, and/or computer-generated image, and/or may correspond to a synthetic, made-up, nonexistent, and/or computer-generated patient (e.g., in other words, the input scan image may not need to be a real scan, or correspond to a real patient). Such a patient and/or image may, for example, be generated by changing one or more medical data, biometric data (e.g. age, ethnicity, etc.), genetic, and/or other datum of an existing patient or a cohort of patient, or by combining data from several real or synthetic patients or cohorts of patient. This may be useful, inter alia, to help overcome data bias, as described above.

As above, computational model 102 may be a single computational model or a plurality of computation models simulating tumor growth. In certain aspects, computational model 102 is a plurality of models where one or more models differ with respect to values of model parameters, initial conditions, boundary conditions, and/or environmental conditions. Computational model 102 may be a binary fluid model (e.g. Cahn-Hilliard), an agent-based model, a molecular dynamics model, a mesh-based model, a cellular automaton model, a finite state machine, a multicomponent model, to name a few. In certain aspects, computational model 102 is used to capture cell proliferation, growth of blood vessels and/or capillaries, nutrient and/or oxygen exchange, energy balance in the simulated tumor (and/or its parts), cancerous cells' invasion of surrounding tissues, and/or the formation of metastases. In certain aspects, computational model 102 is used to capture the levels of various types of ribonucleic acid (RNA), proteins, peptides, and/or other compounds in the body. In certain aspects, computational model 102 is used to capture capturing the genetic makeup, mutations, and/or biomarkers of normal, precancerous, and/or cancerous tissue(s). In certain aspects, computational model 102 is used to capture edema, compression, and/or other effects on the surrounding tissues. In certain aspects, computational model 102 is used to capture the surrounding tissues' (e.g., connective tissue, bone, etc.) effect on the tumor growth. In certain aspects, computational model 102 is used to capture some or all of the effects of therapy and/or treatment.

In certain aspects, computational model 102 may be considered a “context” model that improves the performance of later stage models in a processing pipeline, such as depicted in FIG. 1A. In particular, computational model 102 may process an input scan image of a tumor representing the state of the tumor at a first timestamp to model future states of the tumor at timestamps later in time the first timestamp. For example, computational model 102 may be configured to process a CT scan of a tumor for a patient taken today to predict and model how the tumor may grow and interact with other tissues and/or systems in the body at a time in the future (e.g., a month from today). In certain aspects, computation model 102 is additionally, or alternatively, configured to model previous states of the tumor at timestamps earlier in time than the first timestamp of the input scan image.

In certain aspects, computation model 102 may be configured differently (e.g., using model configurations) such that different simulations of the same or different tumors may be modeled. The model configurations may include one or more model configuration parameters which specify how the model should run, the type of tumor which is to be simulated and/or the location of the tumor which is to be simulated. The model configurations may additionally include a patient's (e.g., a patient with the tumor being simulated) medical data, biometric data, genetic data, epigenetic data, and/or the like. Computational model 102 may take into consideration each of these model configuration parameters when simulating tumor growth for the patient.

According to aspects described herein, different tumor regions may be modeled using computational model 102 to generate synthetic scan images having the particular tumor region identified therein. The different tumor regions that may be modeled by computational model 102 include perifocal edema, tumor margins, metastases, vasculature, necrotic core, hypoxic areas, areas affected by therapy or surgery, etc.

In certain aspects, one or more other computational models are used in additional to computational model 102 in system 100 to simulate tissue movement around a tumor that is being simulated by computational model 102. The tissue movement modeled by the one or more other computational models may be due to pose, heartbeat, breathing, peristalsis, edema, surgical and/or therapeutic procedures, and the like. In certain aspects, information from this simulation may provide additional information (e.g., context) to image adaptation model 106 to generate synthetic scan images and/or predictor model 108 to detect simulated tumor(s). The one or more other computational models may have their own storage, registry, database of models, images, annotations, parameters, data, contours, etc., and/or share some or all of these with computational model 102.

Storage module 104 may be hardware or software configured to capture, register, and store model output images of a tumor simulated by computational model 102. For example, storage module 104 may be an imaging device or other similar component, or software. As illustrated in FIG. 1A, when an input scan image of a tumor is used as input into computational model 102 to simulate growth of the tumor over time, storage module 104 may be configured to capture one or more model output images of the simulated tumor (e.g., as needed) over the period of time. Storage module 104 may store each of these model output images in memory (e.g., volatile or non-volatile memory) and maintain a registry of the images that are stored. The registry may keep a record of memory locations where each of the model output images are kept in memory. In certain other aspects, storage module 104 stores the model output images in an edge device, a fog architecture, the cloud, and/or the like.

In certain aspects, the model output images are captured by storage module 104 at a predetermined time interval, or a user selected time interval. For example, model output images collected by storage module 104 may represent a state of the simulated tumor every 12 hours, every day, every other day, every two weeks, etc. In certain aspects, the model output images captured by storage module 104 are stored as a time-ordered sequence in storage. In certain aspects, storage module 104 encrypts captured model output images prior to storing the images in memory to protect the confidentiality of the image data.

In certain aspects, computational model 102 generates one or more annotations (e.g., contours of the simulated tumor) and/or labels for a simulated tumor. These annotations may be stored alongside each of the model output images in memory. In certain aspects, a clinical expert may manually add annotations to each of the model output images to provide additional context to each of the model output images, for example, as illustrated in FIG. 1A. This additional context may be stored with each of the model output images. Further, storage module 104 may store metadata for each of the model output images together with the annotations and/or labels computational model 102 generates. The metadata may include information about the type of tumor simulated, a patient's medical history, a patient's biometric data, genetic data, epigenetic data, and/or the like. In certain aspects, storage module 104 may read, write, and/or modify 2D and/or 3D images and metadata, jointly or separately, in DICOM, NIFTI, NRRD, MINC, EXIF, SGML, XML, MP4, OGG, or other formats, as appropriate.

In certain aspects, storage module 104 is further configured to retrieve one or more model output image(s) previously stored and provide these retrieved images to an image adaptation model 106. For example, as shown in FIG. 1A, three model output images with timestamps, T=1, T=2, and T=3 may be retrieved by storage module 104 and provided to image adaption model 106. In the example illustrated in FIG. 1A, the retrieved model output images include annotations generated by the computational model 102, generated by a separate model, or manually added to the model output images by, for example, a clinical expert.

Although the example system 100 includes storage module 104, in certain other embodiments, storage module 104 may be omitted. As such, model output image(s) may be provided to image adaptation model 106 through a pipeline, without use of storage module 104.

Image adaptation model 106 is a machine learning model trained to convert model output images to synthetic scan images, with or without annotations. In particular, model output images generated from computational model 102 may be non-realistic images of a simulated tumor with or without annotations. As such, image adaptation model 106 may be configured to convert these non-realistic images to realistic representations of CT scans, PET scans, MRI scans, etc. for the simulated tumor and/or surrounding tissues and structures. As used herein, a synthetic scan image, or pseudo scan image, is an artificial image generated to look like a real-world scan image. According to aspects described herein, the synthetic scan images are artificially generated to represent different states of a tumor (e.g., simulated by computation model 102) at different times over the lifecycle of the tumor. In certain aspects, the synthetic scan images are two-dimensional (2D) and/or three-dimensional (3D) representations of the different states of the simulated tumor. The 2D representations and/or 3D representations may include CT scans, Mill images, fMRI images, PET scans, 3D models, and/or the like. FIG. 1B illustrates an example model output image compared to an example synthetic scan image, according to aspects of the present disclosure.

In certain aspects, image adaptation model 106 is implemented as software, a combination of software and hardware, or by hardware. In certain aspects, image adaptation model 106 is a generative adversarial network (GAN) and/or a diffusion network model, including its interfaces, training and/or testing hardware, software, and/or data. GANs and diffusion networks are an approach to generative modeling using deep learning methods, such as convolutional neural networks. Generative modeling is an unsupervised learning task in machine learning that involves automatically discovering and learning the regularities or patterns in input data (e.g., model output images, as described herein) in such a way that the model can be used to generate and/or output new examples that plausibly could have been drawn from the original dataset.

As illustrated in FIG. 1A, synthetic scan images generated by image adaptation model 106 may be further provided to a predictor model 108. In some embodiments, predictor model 108 is a machine learning model trained to detect simulated tumor(s) in generated synthetic scan images (e.g., generated based on model output images obtained from computational model 102 simulating the tumor(s)). In some embodiments, predictor model 108 is a machine learning model trained to predict the presence of cancer in generated synthetic scan images. In some embodiments, predictor model 108 is a machine learning model trained to perform image segmentation, and more specifically, identify a region of generated synthetic scan images predicted to show/have cancer. Predictor model 108 may comprise one or more similar and/or dissimilar neural network(s) such as a predictor, an autoencoder, an encoder-decoder, a future encoder, and/or the like. In certain aspects, predictor model 108 is implemented as software, a combination of software and hardware, or by hardware.

In certain aspects, predictor model 108 may provide information about detected simulated tumor(s) in the generated synthetic images. The information may include information about properties and/or attributes of the simulated tumor(s), including areas, boundaries, contours, volumes, centers, and/or the like about the simulated tumor(s).

Accordingly, output from predictor model 108 may include one or more synthetic scan images (e.g., with one or more annotations) having simulated tumor(s) detected therein. As described above, these synthetic, annotated scans may be used to (1) supplement available clinic data to better train machine learning models used for cancer/tumor diagnostics and/or treatment (2) guide invasive or noninvasive therapy and/or treatment, and (3) quantify the effect of therapy or treatment for an individual patient or a cohort of patients. The images generated may further aid in pathology detection, monitoring, prognosis, treatment, and/or treatment effect evaluations, in a variety of medical and/or veterinary applications.

According to aspects described herein, to generate synthetic scan images having simulated tumors detected therein, as described with respect to FIG. 1A, at least image adaptation model 106 and predictor model 108 may be trained prior to deployment.

### Example Operations for Training a Tumor Detection System

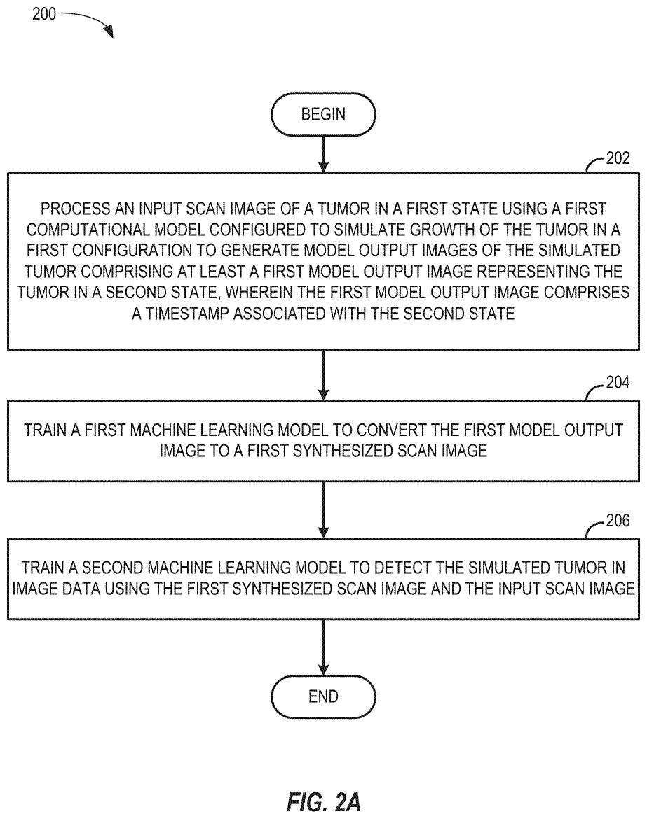

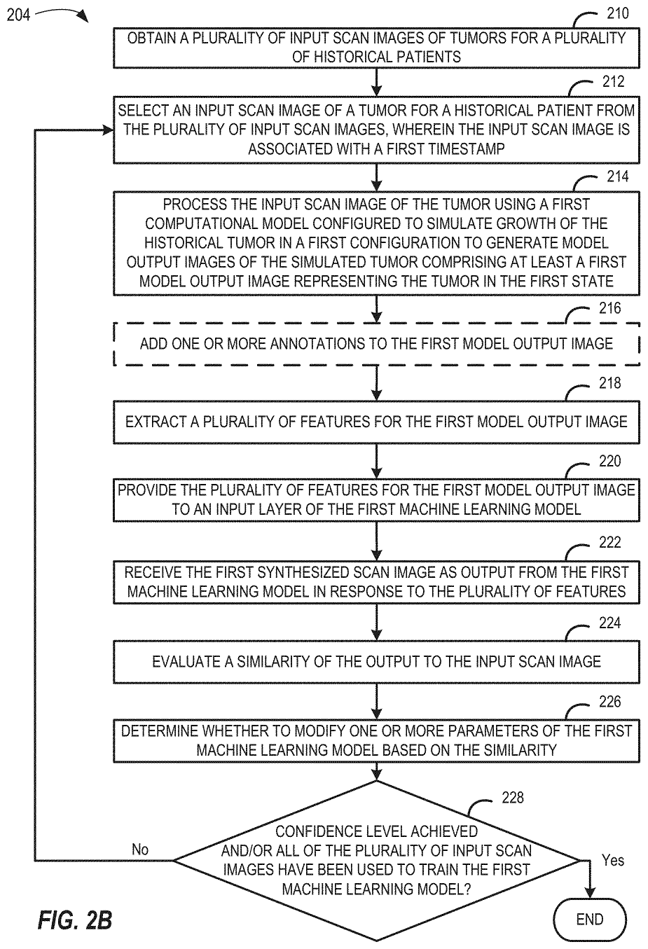

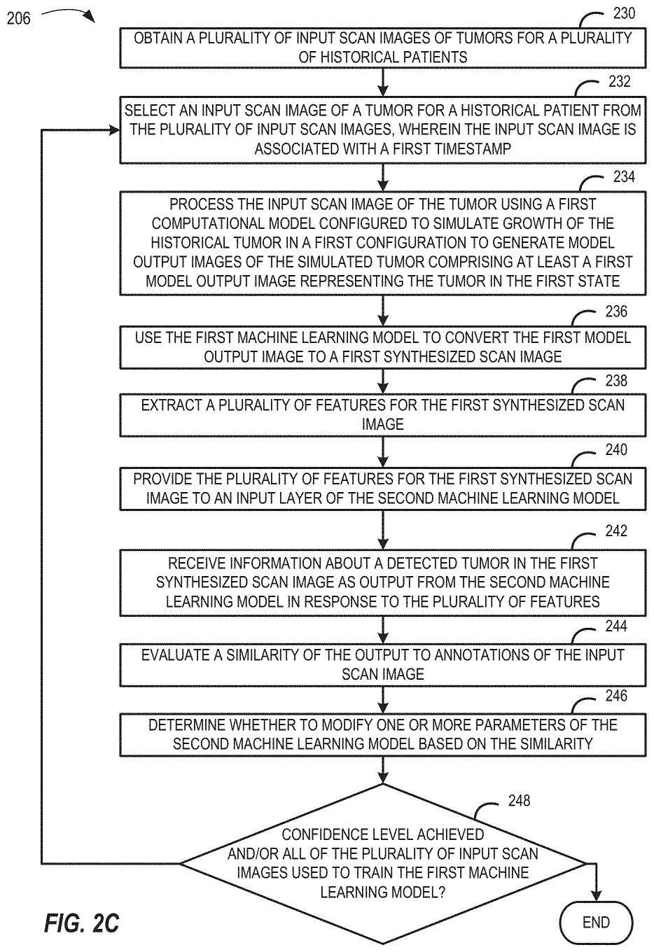

FIG. 2A illustrates example operations 200 for training a tumor detection system, according to aspects of the present disclosure. The tumor detection system may be system 100 illustrated in FIG. 1A.

As illustrated, operations 200 begin at block 202, with processing an input scan image of a tumor in a first state using a first computational model configured to simulate growth of the tumor in a first configuration to generate model output images of the simulated tumor comprising at least a first model output image representing the tumor in a second state, wherein the first model output image comprises a timestamp associated with the second state. As an illustrative example, a CT scan of a tumor for a historical patient may be used as input into a computational model, such as computational model 102 illustrated in FIG. 1A. The CT scan may be a scan that was collected by a clinical expert in October 2022 and thus represents the state of the patient's tumor in October 2022. At block 202, the CT scan of the tumor may be processed using computational model 102 to simulate grown of the tumor to generate model output images of the tumor. The model output images of the tumor may represent different states of the tumor prior in time and/or later in time to October 2022. For example, a first model output image among the model output images may represent a future state (e.g., second state) of the tumor, for example, in October 2023.

At block 204, operations 200 proceed with training a first machine learning model to convert the first model output image to a first synthesized scan image. For example, the first machine learning model may be trained to covert the model output image representing the state of the simulated tumor in October 2023 to a first synthesized scan image representing the state of the simulated tumor in October 2023. In certain aspects, the first machine learning model comprises image adaptation model 106 illustrated in FIG. 1A.

Techniques for training the first machine learning model are described in detail in FIG. 2B. In particular, FIG. 2B illustrates example operations 204 for training the first machine learning model (e.g., image adaptation model 106) to convert computational model output to synthesized scan image(s), according to aspects of the present disclosure. Operations 204 may be performed by a computing system (e.g., physical or virtual), such as a model training engine.

As illustrated, operations 204 begin at block 210, with obtaining a plurality of input scan images of tumors for a plurality of historical patients. The inputs scan images may include a combination of X-rays, CT scans, PET scans, MRI scan, and/or fMRI scans available for specific pathologies. The input scan images may include scans representing states of different tumor in their earliest stages, during avascular growth, during vascular growth, and/or during metastasis. The input scan images may be scan images collected for a variety of individuals of different sex, gender, age, and/or the like. Further, the input scan images may represent tumors for different historical patients that engaged in one or more forms of therapy/treatment and/or did not engage in any therapy/treatment. Each of the input scan images may include one or more annotations (e.g., previously provided by clinical experts) identifying when the scan image was taken, whether the tumor exists in the scan image, a location of the tumor, a size of the tumor, an indication of how the tumor has affected nearby tissue, etc. The plurality of input scan images obtained at block 210 may be representative of training data used to train the first machine learning model.

At block 212, operations 204 proceed with selecting an input scan image of a tumor for a historical patient from the plurality of input scan images. The input scan image may be associated with a first timestamp. In other word, at block 212, a first training data instance is selected and provided to the model training engine. As an illustrative example, the first training data instance may be a PET scan for a historical patient representing the state of the patient's tumor one year prior (e.g., a fist state).

At block 214, operations 204 proceed with processing the input scan image of the tumor using computational model 102 (e.g., illustrated in FIG. 1A) to simulate growth of the historical tumor in a first configuration. Simulation of the historical tumor may generate one or more model output images of the simulated tumor. The one or more model output images may include at least a first model output image of the simulated tumor representing the state of the tumor one year prior (e.g., the first state).

At block 216, operations 204 optionally proceed with adding one or more annotations to the first model output image. In certain aspects, the one or more annotations are manually added by a clinical expert to provide additional context to the first model output image. In certain aspects, the one or more annotations are generated by a separate model.

At block 218, operations 204 proceed with extracting a plurality of features for the first model output image. Each feature, may represent a measurable piece of data that can be used for analysis. The plurality of features extracted for this first model output image may include the tumor's boundaries, size, volume, location, vasculature, perifocal edema, necrotic core, locations and/or boundaries of affected and non-affected organs and structures, etc. Operations 204 then proceed to block 220, where the plurality of features for the first model output images are provided to an input layer of the first machine learning model.

At block 222, operations 204 proceed with receiving output from the first machine learning model (e.g., image adaptation model 106 illustrated in FIG. 1A) in response to the plurality features. In certain aspects, the model training engine receives an inference as an output from the first machine learning model. The inference may be output by the final layer or output layer of the first machine learning model. The inference may be a predicted synthesized scan image meant to represent the first state of the simulated tumor. Using the previous example, the predicted synthesized scan image, generated by the first machine learning model, may be generated to represent the historical tumor for the patient one year prior.

At block 224, operations 204 proceed with evaluating a similarity of the output to the input scan image selected at block 212. For example, the synthesized scan image generated to represent the historical tumor for the patient one year prior may be compared against the real input scan image representing the state of the patient's tumor one year prior. In certain aspects, evaluating the similarity of the output to the input scan image is performed using a loss function.

At block 226, operations 204 proceed with determining whether to modify one or more parameters of the first machine learning model based on the similarity determined at block 224. In certain aspects, the model training engine trains the first machine learning model by iteratively modifying parameters (e.g., through backpropagation) of the first machine learning model until its output in response to the training input matches the training output. For example, the output may be compared to the training output (e.g., at block 224), and various parameters such as weights, connections, number of hidden nodes, weight decay, activation sparsity, nonlinearity, weight initialization, random seeds, model averaging, preprocessing input data, coordinate descent, grid search, random search, and model-based optimization methods, may be modified such until the output matches the training output.

In certain aspects, this process is performed for a plurality of training data instances in order to train the first machine learning model. For example, at block 228, operations 204 proceed with determining whether a confidence level has been achieved and/or whether all of the plurality of input scan images have been used to train the first machine learning model. In particular, a confidence score that represent the likelihood that the output of the first machine learning model is correct and will satisfy a user's request is calculated. This confidence level may be calculated based on the similarity of the output to the input scan. Where the confidence level is above a threshold confidence level (e.g., predetermined and/or selected by a user), training of the first machine learning model may be complete. Additionally, or alternatively, the model training engine may determine whether all of the plurality of input scan images (e.g., collected at block 210) have been used to train the first machine learning model. Where the confidence level is below the threshold and/or all of the plurality of input scan image have not yet been used to train the first machine learning model, operations 204 proceed back to block 212 to select another input scan image for training the first machine learning model.

Referring back to FIG. 2A, after the first machine learning model has been trained to convert the first model output image to a first synthesized scan image, at block 206, operations 200 proceed with training a second machine learning model to detect the simulated tumor in image data using the first synthesized scan image and the input scan image. For example, the second machine learning model may be trained to detect the simulated tumor in the first synthesized scan image representing the state of the simulated tumor in October 2023. In certain aspects, the second machine learning model comprises predictor model 108 illustrated in FIG. 1A.

Techniques for training the second machine learning model are described in detail in FIG. 2C. In particular, FIG. 2C illustrates example operations 206 for training a machine learning model to detect a simulated tumor in a synthesized scan image, according to aspects of the present disclosure. Operations 206 may be performed by a computing system (e.g., physical or virtual), such as a model training engine.

As illustrated, initial operations 206 (e.g., at block 230, 232, and 234) for training the second machine learning model (predictor model 108 illustrated in FIG. 1A) are similar to the initial operations (e.g., at block 210, 212, and 214) 204, illustrated in FIG. 2B, used to train the first machine learning model. In particular, operations 206 begin at block 230 with obtaining a plurality of input scan images of tumors for a plurality of historical patients. At block 232, operations 206 proceed with selecting an input scan image of a tumor for a historical patient from the plurality of input scan images. Further, at block 234, operations 206 proceed with processing the input scan image of the tumor using computational model 102 (e.g., illustrated in FIG. 1A) to simulate growth of the historical tumor in a first configuration. Simulation of the historical tumor may generate one or more model output images of the simulated tumor. The one or more model output images may include at least a first model output image of the simulated tumor representing the state of the tumor one year prior (e.g., the first state).

At block 236, operations 206 proceed with converting the first model output image to a first synthesized scan image using the first machine learning model. At block 238, operations 206 proceed with extracting a plurality of features for the first synthesized scan image. Operations 206 then proceed to block 240, where the plurality of features for the first synthesized scan image are provided to an input layer of the second machine learning model.

At block 242, operations 206 proceed with receiving output from the second machine learning model in response to the plurality features. In certain aspects, the model training engine receives an inference as an output from the first machine learning model. The inference may be output by the final layer or output layer of the second machine learning model about a detected tumor in the first synthesized scan image. Using the previous example, the detected tumor in the synthesized scan image may be the simulated tumor (e.g., simulated via computational model 102) representing the historical tumor for the patient one year prior.

At block 244, operations 206 proceed with evaluating a similarity of the output to the input scan image selected at block 232. For example, the simulated tumor detected by the second machine learning model in the first synthesized scan image may be compared against annotations in the real input scan image (e.g., made by a clinical expert) identifying the tumor in the real input scan image. At block 246, operations 206 proceed with determining whether to modify one or more parameters of the first machine learning model based on the similarity determined at block 244. In certain aspects, evaluating the similarity of the output to the input scan image selected at block 232 is performed using a loss function.

In certain aspects, operations 206 are performed for a plurality of training data instances in order to train second machine learning model (predictor model 108 illustrated in FIG. 1A). For example, at block 248, operations 206 proceed with determining whether a confidence level has been achieved and/or whether all of the plurality of input scan images have been used to train the first machine learning model. Where the confidence level is above a threshold confidence level (e.g., predetermined and/or selected by a user), training of the second machine learning model may be complete. Additionally, or alternatively, the model training engine may determine whether all of the plurality of input scan images (e.g., collected at block 230) have been used to train the second machine learning model. Where the confidence level is below the threshold and/or all of the plurality of input scan images have not yet been used to train the second machine learning model, operations 206 proceed back to block 232 to select another input scan image for training the second machine learning model.

In certain aspects, the second machine learning model, or parts thereof, may be trained using self-supervision (e.g., predicting future inputs in part, or in full). Self-supervised learning is a machine learning approach where the model trains itself by leveraging one part of the data to predict the other part and generate labels accordingly. In certain aspects, the second machine learning model, or part thereof, may be trained using transfer learning (e.g., pre-training on data from a publicly and/or commercially available dataset).

### Example Synthetic Data Generation and Annotation Using a Model Trained to Predict Computational Model Output Images

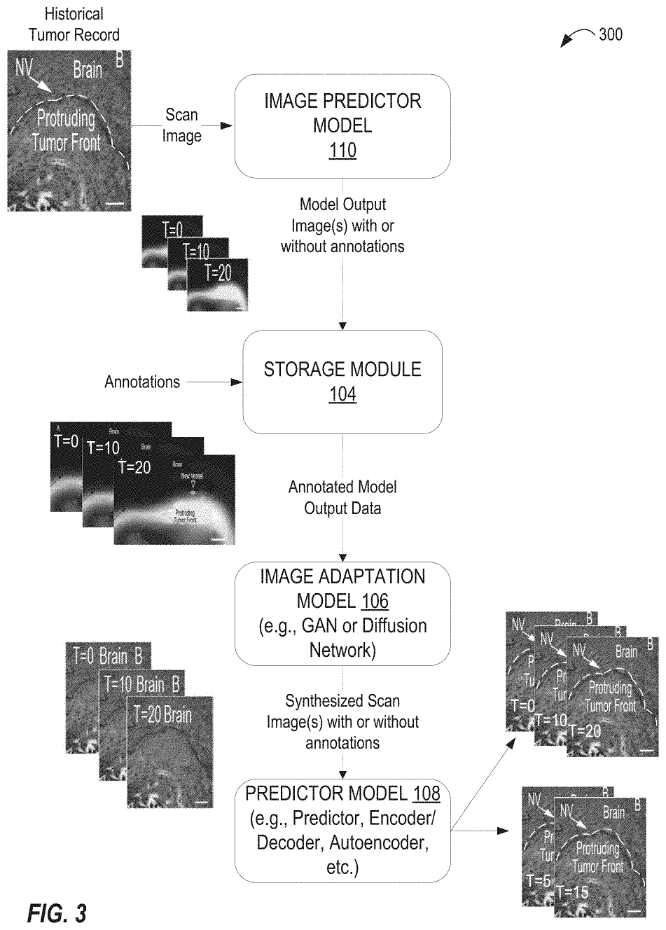

FIG. 3 illustrates another example system 300 for synthetic data generation and diagnostic prediction (e.g., tumor detection in this example), according to certain aspects of the present disclosure. Example system 300 includes an image predictor model 110, a storage module 104, an image adaptation model 106, and a predictor model 108, as described above with respect to FIG. 1A. As described above, in certain aspects, an image predictor model 110 may be used in place of computational model 102. Accordingly, power and/or resources may be saved when generating the annotated, synthetic scan images given the computational model 102 is not needed to simulate tumor growth each time synthesized scan images are to be generated for a different tumor and/or tumor configuration.

Image predictor model 110 is a machine learning model configured to predict and generate predicted model output images for a simulated tumor. For example, image predictor model 110 may be trained to predict and generate predicted model output images at past, current, and/or future time points for a simulated tumor given the parameters and the initial or boundary conditions (as well as other pertinent data, such as biometrics or genomics) provided to the model. Image predictor model 110 may be trained to predict and generate such images without having to actually perform simulation of the tumor via computational model 102.

In certain aspects, image predictor model 110 is configured to predict and generate predicted model output images of a simulated tumor at a predetermined time step (or at a selected time step) such that a plurality of predicted model output images of the simulated tumor are generated and represent different state of the tumor for the time period. For example, as illustrated in FIG. 3, image predictor model 110 may be configured to generate predicted model outputs based on a ten day time interval. Thus, predicted model output images generated by image predictor model 110 may include a predicted model output image representing a current state of a simulated tumor (e.g., at T=0), a predicted model output image representing a state of the tumor ten days in the future (e.g., at T=10), and a predicted model output image representing a state of the tumor twenty days in the future (e.g., at T=20). Thus, although image predictor model 110 may reduce an amount of resources and/or power used to generate model output images of a simulated tumor, an amount and/or granularity of model output images generated over a period of time may be less than model output images generated where a computational model 102 is used to simulate tumor growth.

Similar to FIG. 1A, these predicted model output images may be registered and stored in memory (and/or on an edge device, a fog architecture, the cloud, and/or the like). In certain aspects, image predictor model is further trained to generate one or more annotations (e.g., contours of the simulated tumor) and/or labels for the simulated tumor. These annotations may be stored in relation to each of the predicted model output images in memory. In certain aspects, a clinical expert may manually add annotations to each of the predicted model output images to provide additional context to each of the predicted model output images, for example, as illustrated in FIG. 3. This additional context may be stored with each of the model output images. Further, storage module 104 may store metadata for each of the model output images together with the annotations and/or labels image predictor model 110 generates. The metadata may include information about the type of tumor simulated, a patient's medical history, a patient's biometric data, genetic data, epigenetic data, and/or the like.

Storage module 104 is further configured to retrieve one or more model output image(s) previously stored and provide these retrieved images to an image adaptation model 106. For example, storage module 104 may retrieve the predicted model output image representing a current state of the simulated tumor (e.g., at T=0), the predicted model output image representing a state of the simulated tumor ten days in the future (e.g., at T=10), and a predicted model output image representing a state of the simulated tumor twenty days in the future (e.g., at T=20), and provide these images to image adaptation model 106, as illustrated in FIG. 3.

Although the example system 300 includes storage module 104, in certain other embodiments, storage module 104 may be omitted. As such, model output image(s) predicted by image predictor 110 may be provided to image adaptation model 106 through a pipeline, without use of storage module 104.

Similar to FIG. 1A, image adaptation model 106 in FIG. 3 may be trained to convert the predicted model output images to synthetic scan images (e.g., referred to herein as “first synthesized scan images”), with or without annotations, and the generated synthetic scan images may be subsequently provided to a predictor model 108.

In certain aspects, similar to FIG. 1A, predictor model 108 in FIG. 3 is a machine learning model trained to detect simulated tumor(s) in generated synthetic scan images (e.g., generated based on model output images obtained predicted/generated by image predictor model 110).

In certain other aspects, predictor model 108 is trained to generate additional synthetic scan images (e.g., referred to herein as “second synthesized scan images”). The additional synthetic scan images generated by predictor model 108 may represent a state of the simulated tumor in a state in time between at least two of the plurality of synthetic scan images. In other words, the second machine learning mode may be trained to perform interpolation to generate one or more synthetic scan images. For example, as illustrated in FIG. 3, in certain aspects, predictor model 108 uses the predicted model output image at T=0 and the predicted model output image at T=10 to generate one second synthetic scan image representing the simulated tumor five days in the future (e.g., T=5). Further, predictor model uses the predicted model output image at T=10 and the predicted model output image at T=20 to generate another second synthetic scan image representing the simulated tumor fifteen days in the future (e.g., T=15). Thus, predictor model 108 may be used to “fill in the gaps” where model output images image predictor model 110 generates model output images with a larger time interval. As such, predictor model 108 may be a bi-directional predictor, or in other words, a machine learning model capable of predicting synthetic scan images and/or data at earlier, later, and/or intermediate time points relative to the synthetic scan images (e.g., having different timestamps) provided as input into predictor model 108. Predictor model 108 may use machine learning architectures such as, for example, a transformer network, networks based on uni-directional or bi-directional long short-term memory (LSTM), gated recurrent unit (GRU), other recurrent and/or stateful units and components, and/or the like.

In certain aspects, predictor model 108 generates additional synthetic scan images and detects simulated tumor(s) in the additional synthetic scan images. Accordingly, similar to FIG. 1A, output from predictor model 108 may include one or more synthetic scan images (e.g., with one or more annotations) having simulated tumor(s) detected therein.

In certain aspects, image predictor model 110 and image adaptation model 106 may learn by implementing adversarial learning algorithms (e.g., as a GAN), for example, with one model learning to distinguish between model output images and synthetic scan images generated by the other model. The goal in such adversarial learning is to make the synthetic scan images as close (e.g., that is, as indistinguishable) as possible to the model output images.

In certain aspects, one or more computational models are additionally used in system 300 to simulate tissue movement around a tumor for which image predictor model 110 is generating predicted model outputs for. The tissue movement modeled by the one or more computational models may be due to pose, heartbeat, breathing, peristalsis, edema, surgical and/or therapeutic procedures, and the like. In certain aspects, information from this simulation may provide additional information (e.g., context) to image adaptation model 106 to generate synthetic scan images and/or predictor model 108 to generate additional synthetic scan images and/or detect simulated tumor(s).

According to aspects described herein, to generate synthetic scan images having simulated tumors detected therein, as described with respect to FIG. 3, at least image predictor model 110, image adaptation model 106, and predictor model 108 may be trained prior to deployment.

### Example Operations for Training Another Tumor Detection System

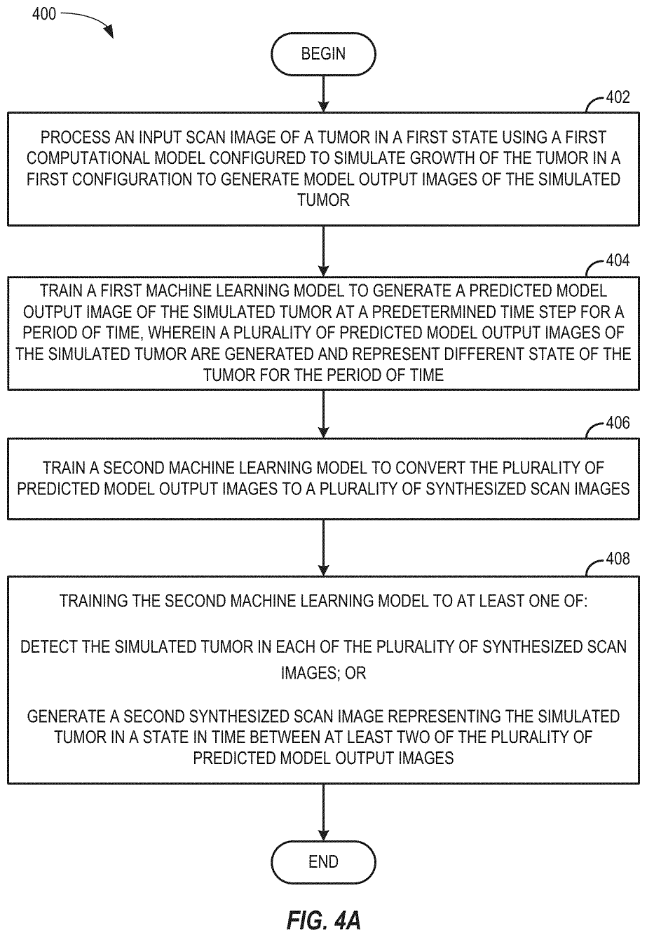

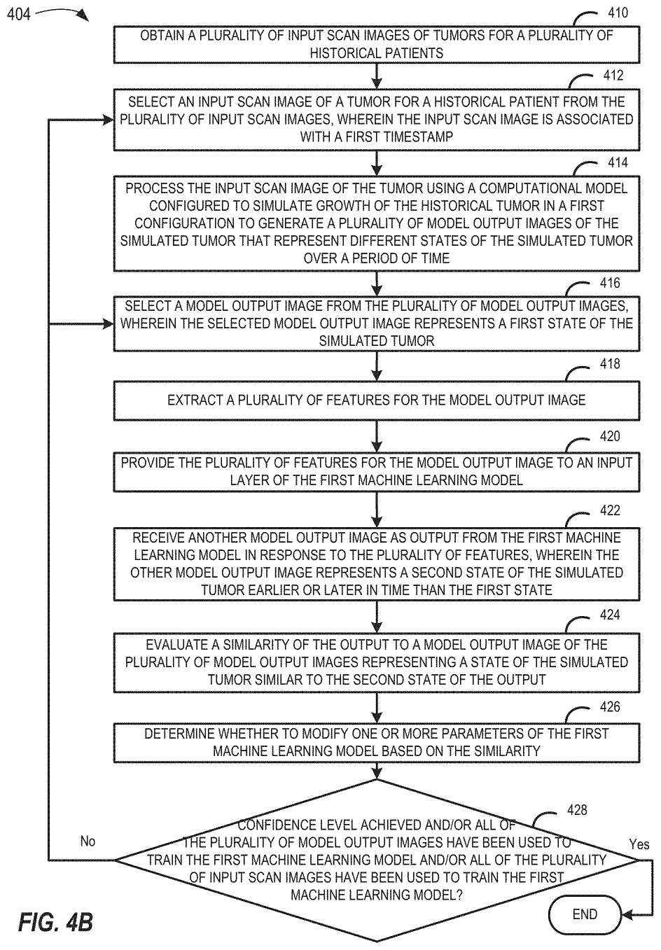

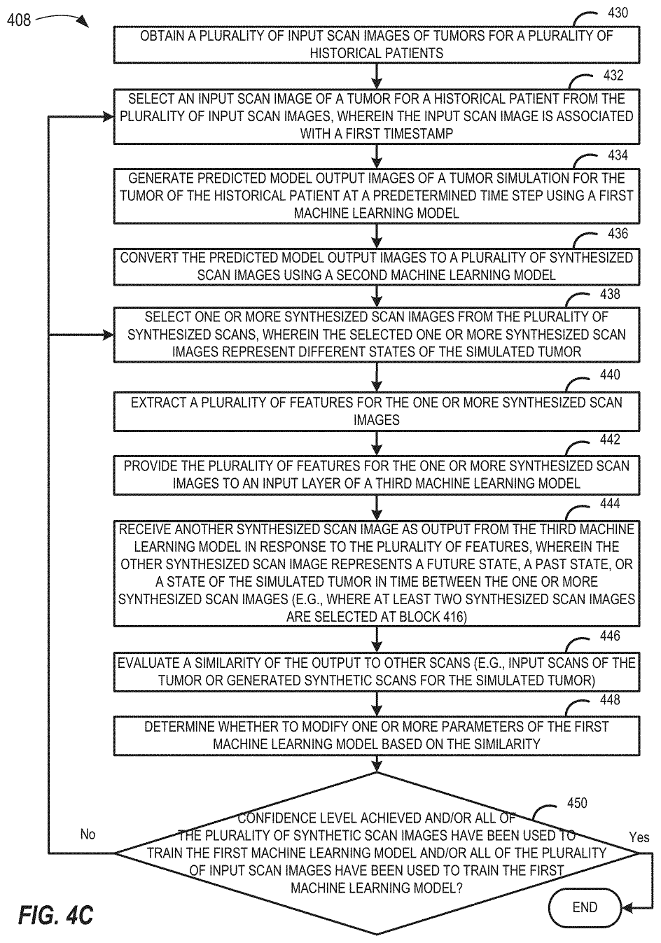

FIG. 4A illustrates example operations 400 for training a tumor detection system, according to aspects of the present disclosure. The tumor detection system may be system 300 illustrated in FIG. 3.

As illustrated, operations 400 begin at block 402, with processing an input scan image of a tumor in a first state using a first computational model configured to simulate growth of the tumor in a first configuration to generate model output images of the simulated tumor. At block 202, model output images of the simulated tumor may be generated. The model output images of the tumor may represent different states of the tumor over time.

At block 404, operations 400 proceed with training a first machine learning model to generate a predicted model output image of the simulated tumor at a predetermined time step for a period of time. Accordingly, at block 404, the first machine learning model is trained to generate a plurality of predicted model output images of the simulated tumor, where each predicted model output images represents a different state of the tumor for the period of time. In certain aspects, the first machine learning model comprises image predictor model 110 illustrated in FIG. 3.

Techniques for training the first machine learning model are described in detail in FIG. 4B. In particular, FIG. 4B illustrates example operations 404 for training the first machine learning model (e.g., image predictor model 110 of FIG. 1A) to generate predicted model output image(s) representing different states of a simulated tumor over a period of time, according to aspects of the present disclosure. Operations 404 may be performed by a computing system (e.g., physical or virtual), such as a model training engine.

As illustrated, initial operations 404 (e.g., at block 410, 412, and 414) for training the first machine learning model (e.g., image predictor model 110 of FIG. 1A) in FIG. 3 are similar to the initial operations illustrated in FIGS. 2A and 2B (e.g., at blocks 210, 212, 214, 230, 232, and 234, respectively). In particular, operations 404 begin at block 410 with obtaining a plurality of input scan images of tumors for a plurality of historical patients. At block 412, operations 404 proceed with selecting an input scan image of a tumor for a historical patient from the plurality of input scan images. Further, at block 414, operations 404 proceed with processing the input scan image of the tumor using a computational model to simulate growth of the historical tumor in a first configuration. Simulation of the historical tumor may generate one or more model output images of the simulated tumor.

At block 416, operations 404 proceed with selecting a model output image from the plurality of model output images. The selected model output image may representing a first state of the simulated tumor at a first point in time. In certain aspects, the selection of the first model output image may be random.

At block 418, operations 404 proceed with extracting a plurality of features for the first model output image. Operations 404 then proceed to block 420, where the plurality of features for the first model output image are provided to an input layer of the first machine learning model.

At block 422, operations 404 proceed with receiving output from the first machine learning model in response to the plurality features. In certain aspects, the model training engine receives an inference as an output from the first machine learning model. The inference may be a predicted model output image. The predicted model output image may represent a second state of the simulated tumor at a second point in time.

At block 424, operations 404 proceed with evaluating a similarity of the output to a model output image of the plurality of model output images (e.g., generated at block 414) representing a state of the simulated tumor similar to the second state of the output. For example, where the predicted model output image of the simulated tumor (e.g., received as output form the first machine learning model at block 422) represents a state of the simulated tumor in October 2022, then the received model output image may be compared to a model output image (e.g., generated at block 414) that also represents a state of the simulated tumor in October 2022 (although the selected model output image represents a state of the simulated tumor at a different point in time, for example, in January 2022).

At block 426, operations 404 proceed with determining whether to modify one or more parameters of the first machine learning model based on the similarity determined at block 424.

At block 428, operations 404 proceed with determining (1) whether a confidence level has been achieved for the first machine learning model, (2) whether the plurality of model output images (e.g., generated at block 414) have been used to train the first machine learning model, and/or (3) whether all of the plurality of input scan images have been used to train the first machine learning model. In certain aspects, if a confidence level has been achieved, training the first machine learning model may be complete. In certain aspects, if the confidence level has not been achieved, but all of the plurality of model output images have been used to train the first machine learning model and all of the plurality of input scan images have been used to train the first machine learning model, training the first machine learning model may be complete. In certain aspects, if all of the plurality of model output images have been used to train the first machine learning model and all of the plurality of input scan images have been used train the first machine learning model, training the first machine learning model may be complete. Otherwise, in the above scenarios, operations 404 may return to block 416 (e.g., when all of the plurality of model output images have not been used to train the first machine learning model) or to block 412 (e.g., when all of the plurality of input scan images have not been used train the first machine learning model).

Referring back to FIG. 4A, after the first machine learning model has been trained to generate predicted model output image(s) representing different states of a simulated tumor over a period of time, at block 406, operations 400 proceed with training a second machine learning model to convert predicted model output images (e.g., predicted and generated by the first machine learning model, image predictor model 110) to first synthesized scan images. In certain aspects, the second machine learning model comprises image adaptation model 106 illustrated in FIG. 3. Techniques for training the second machine learning model are similar to the techniques previously described with respect to FIG. 2B.

At block 408, operations 400 proceed with training a third machine learning model. In certain aspects, the third machine learning model comprises predictor model 108 illustrated in FIG. 3.

In certain aspects, the third machine learning model is trained to detect the simulated tumor in each of the plurality of synthesized scan image (e.g., generated by the second machine learning model). Techniques for training the third machine learning model to detect the simulated tumor(s) are similar to the techniques previously described with respect to FIG. 2C.

Additionally or alternatively, the third machine learning model is trained to generate a second synthesized scan image representing the simulated tumor in a state in time between at least two of the plurality of predicted model output images (e.g., generated by image predictor model 110). Techniques for training the third machine learning model are described in detail in FIG. 4C. In particular, FIG. 4C illustrates example operations 408 for training a machine learning model to generate synthesized scan image(s) representing a future and/or past state of a tumor, according to aspects of the present disclosure. Operations 408 may be performed by a computing system (e.g., physical or virtual), such as a model training engine.

As illustrated, initial operations 408 (e.g., at block 430 and 432) for training the third machine learning model (e.g., predictor model 108) in FIG. 3 are similar to the initial operations illustrated in FIGS. 2A, 2B, and 4B (e.g., at blocks 210, 212, 230, 232, 410, and 412, respectively). In particular, operations 408 begin at block 430 with obtaining a plurality of input scan images of tumors for a plurality of historical patients. At block 432, operations 408 proceed with selecting an input scan image of a tumor for a historical patient from the plurality of input scan images.

At block 434, operations 408 proceed with generating predicting model output images of at tumor simulation for the tumor of the historical patient at a predetermined time step using a first machine learning model (e.g., image predictor model 110 illustrated in FIG. 3). For example, in a first example, image predictor model may generate three predicted model output images including (1) a predicted model output image representing a state of a simulated tumor on Jan. 1, 2022 (e.g., at T=0), (2) a predicted model output image representing a state of the tumor ten days later (e.g., from Jan. 1, 2022) on Jan. 10, 2022 (e.g., at T=10), and (3) a predicted model output image representing a state of the tumor twenty days later (e.g., from Jan. 1, 2022) on Jan. 20, 2022 (e.g., at T=20). Where a time step for generating predicted images is smaller in a second example, image predictor model may generate five predicted model output images including (1) a predicted model output image representing a state of a simulated tumor on Jan. 1, 2022 (e.g., at T=0), (2) a predicted model output image representing at state of the simulated tumor five days later (e.g., from Jan. 1, 2022) on Jan. 5, 2022 (e.g., T=5), (3) a predicted model output image representing a state of the simulated tumor ten days later (e.g., from Jan. 1, 2022) on Jan. 10, 2022 (e.g., at T=10), (4) a predicted model output image representing a state of the simulated tumor fifteen days later (e.g., from Jan. 1, 2022) on Jan. 15, 2022 (e.g., at T=15), and (5) a predicted model output image representing a state of the simulated tumor twenty days later (e.g., from Jan. 1, 2022) on Jan. 20, 2022 (e.g., at T=20).

At block 436, operations 408 proceed with converting the predicted model output images a plurality of synthesized scan images using a second machine learning model (e.g., image adaptation model 106 illustrated in FIG. 3). For example, using the second example above, the second machine learning model may convert the five predicted model output images to five synthesized scan images.

At block 438, operations 408 proceed with selecting one or more synthesized scan images from the plurality of synthesized scans. The selected one or more synthesized scan images may represent different state of the stimulated tumor (and be associated with different timestamps). For example, among the five synthesized scan images, two synthesized scan images may be selected at block 438. In particular, a synthesized scan image representing the state of the simulated tumor on Jan. 1, 2022 (e.g., at T=0) and a synthesized scan image representing the state of the simulated tumor on Jan. 10, 2022 (e.g., at T=10) may be selected.

At block 440, operations 408 proceed with extracting a plurality of features for the selected synthesized scan image(s) (e.g., the two selected synthesized scan images). Operations 408 then proceed to block 442, where the plurality of features for the synthesized scan image(s) are provided to an input layer of a third machine learning model (e.g., predictor model 108 illustrated in FIG. 3).

At block 444, operations 408 proceed with receiving output from the first machine learning model in response to the plurality features. In certain aspects, the model training engine receives an inference as an output from the first machine learning model. The inference may be another generated synthesized scan image. In certain aspects, the other synthesized scan image represents a future state of the simulated tumor. In certain aspects, the other synthesized scan image represents a past state of the simulated tumor. In certain aspects, the other synthesized scan image represents a state of the simulated tumor in time between the synthesized scan images, where at least two synthesized scan images were selected at block 416. For example, using the previous example, the output received at block 444 may be a synthesized scan image (e.g., predicted and generated by the third machine learning model) representing a state of the simulated tumor on Jan. 5, 2022 (e.g., at T=5) (e.g., which is a state of the simulated tumor that is between the first synthesized scan (e.g., on Jan. 1, 2022 at T=0) and the second synthesized scan (e.g., on Jan. 10, 2022 at T=10) used as input into the third machine learning model).

At block 446, operations 408 proceed with evaluating a similarity of the output to other scans. In certain aspects, the other scans include an input scan of the tumor for the patient representing the state of the tumor of the historical patient on Jan. 1, 2022, where this input scan exists. In certain aspects, the other scans include the synthesized scan image representing the state of the simulated tumor of Jan. 5, 2022, that was generated by the second machine learning model (e.g., image adaptation model 106) at block 436.

At block 448, operations 408 proceed with determining whether to modify one or more parameters of the first machine learning model based on the similarity determined at block 424.

At block 450, operations 408 proceed with determining (1) whether a confidence level has been achieved for the first machine learning model, (2) whether the plurality of model output images (e.g., generated at block 414) have been used to train the first machine learning model, and/or (3) whether all of the plurality of input scan images have been used to train the first machine learning model. In certain aspects, if a confidence level has been achieved, training the third machine learning model may be complete. In certain aspects, if the confidence level has not been achieved, but all of the plurality of synthetic scan images have been used to train the third machine learning model and all of the plurality of input scan images have been used to train the third machine learning model, training the third machine learning model may be complete. In certain aspects, if all of the plurality of synthetic scan images have been used to train the third machine learning model and all of the plurality of input scan images have been used train the third machine learning model, training the third machine learning model may be complete. Otherwise, in the above scenarios, operations 408 may return to block 438 (e.g., when all of the plurality of synthetic scan images have not been used to train the third machine learning model) or to block 432 (e.g., when all of the plurality of input scan images have not been used train the third machine learning model).

In certain aspects, one or more of the computational models and/or machine learning models described herein may use additional, continuous, and/or lifelong learning when deployed. In particular, these computational model(s) and/or machine learning model(s) may use the data and/or inputs collected and/or provided in deployment, and/or any inputs provided by clinical experts (or other skilled professionals) to continuously learn and adjust their models.

In certain aspects, one or more of the machine learning models may utilize federated learning. For example, when information is learned and/or machine learning model parameters (e.g., weights) are adjusted/improved by one deployed instance (e.g., one machine learning model described herein), this information may be made available to one or more other deployed machine learning models described herein.

In certain aspects, one or more of the computational models and/or machine learning models described herein may be implemented, deployed, trained, maintained, monitored, and/or modified via a model interface. For example, in certain aspects, the model interface received data, which may be filtered through an input filter, through a data input pipeline. The input filter may filter the input data, for example, to remove data that may not be relevant to a given model, to remove duplicative data, to sample the data provided to the model interface, and/or the like. The model interface may pass the input to one or more of the computational models and/or machine learning models for processing. In certain aspects, the model interface may implement additional functionality for modifying a data set, adjusting one or more of the machine learning models (e.g., adjusting parameters of these models), load balancing, and other operations that may be performed to manage operations with respect to these models.

In some aspects, a context wrapper may perform a model parameter search, either adaptive or using a predetermined mesh. This may be used to predict, interpolate, and extrapolate model behavior at arbitrary parameter values. Boundaries on allowed values of parameters may be provided to the context wrapper via various interfaces, such as a command line interface, a graphical user interface, a programmatic interface, an application programming interface (API), and/or the like. Fixed user parameters, which may include parameters that are not identified by the context wrapper 100, can likewise be provided as input to the context wrapper.

In some aspects, the computational model (e.g., computational model 102) serves as a context model, and the first machine learning model (e.g., image adaptation model 106) serves as context extractor/encoder.

### Example Processing System for Stratifying Data Samples for Use in Machine Learning

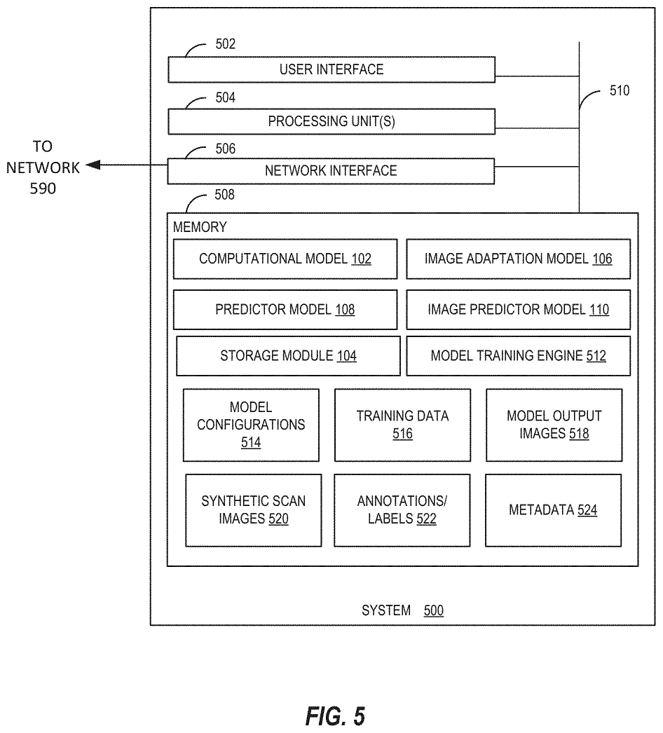

FIG. 5 illustrates an example processing system 500 configured to perform the methods described herein, including, for example, operations of FIGS. 1A-4C. In some embodiments, system 500 may act as a computing system on which a one or more computational models and/or machine learning models may be used in combination to generate synthetic representations of past, present, and/or future predicted states of a tumor.

As shown, system 500 includes a user interface 502, one or more processing units (e.g., a CPU, GPU, TPU, machine learning accelerator, and/or the like) 504, a network interface 506 through which system 500 is connected to network 590 (which may be a local network, an intranet, the internet, or any other group of computing devices communicatively connected to each other), and a memory 508, connected via an interconnect 510.

User interface 502 is configured to provide a point at which users may be able to interact with system 500. User interface 402 may allow users to interact with system 500 in a natural and intuitive way. In certain aspects, a user (e.g., a clinical expert) may interact with system 500 to provide annotations to model output images and/or generated synthetic scan images, as described herein. In certain aspects, user interface 502 is a graphical user interface which allows users to interact with system 500 through interactive visual components.

Processing unit(s) 504 may retrieve and execute programming instructions stored in the memory 408. Similarly, the processing unit(s) 504 may retrieve and store application data residing in the memory 508. The interconnect 510 transmits programming instructions and application data, among the processing unit(s) 504, network interface 506, and memory 508.

In certain aspects, processing unit(s) 504 is included to be representative of a single CPU/GPU/TPU/machine learning accelerator, multiple CPUs/GPUs/TPUs/machine learning accelerators, a single CPU/GPU/TPU/machine learning accelerator having multiple processing cores, and the like.

Memory 508 is representative of a volatile memory, such as a random access memory, or a nonvolatile memory, such as nonvolatile random access memory, phase change random access memory, or the like. As shown, memory 508 includes a computational model 102, an image adaptation model 106, a predictor model 108, an image predictor model 110, a storage module 104, and a model training engine 512. Further, in certain aspects, memory 508 contains data such as model configurations 514, training data 516, model output image 518, synthetic scan images 520, annotations/labels 522, and metadata 524.

In certain aspects, computational model 102 is configured to simulate tumor growth for a specific tumor type (e.g., glioblastoma) and/or a plurality of tumor types, such as described above with respect to FIG. 1A. In particular, computational model 102 may model tumor progression using one or more input scan images.

In certain aspects, image adaptation model 106 is trained to convert model output images to synthetic scan images, with or without annotations, such as described above with respect to FIG. 1A and FIG. 3.

In certain aspects, predictor model 108 is trained to detect simulated tumor(s) in generated synthetic scan images, such as described above with respect to FIG. 1A. In certain aspects, predictor model 108 is trained to generate synthetic scan images for a simulated tumor, such as described above with respect to FIG. 3. The generated synthetic scan images may represent a future state of the simulated tumor, a past state of the simulated tumor, and/or a state of the simulated tumor in time between at least two other synthetic scan images representing different states of the simulated tumor.

In certain aspects, image predictor model 110 is configured to predict and generate predicted model output images of a simulated tumor at a predetermined time step (or at a selected time step) such that a plurality of predicted model output images of the simulated tumor are generated and represent different state of the tumor for the time period, such as described above with respect to FIG. 3.

In certain aspects, storage module 104 is configured to capture, register, and store model output images of a tumor simulated by computational model 102, such as described above with respect to FIG. 1A and FIG. 3.

In certain aspects, model training engine 512 is configured to train image adaptation model 106, predictor model 108, and/or image predictor model 110, such as described above with respect to FIG. 2B, FIG. 2C, FIG. 4B, and FIG. 4C.

Note that FIG. 5 is just one example of a processing consistent with aspects described herein, and other processing systems having additional, alternative, or fewer components are possible consistent with this disclosure.

In certain embodiments, more than one apparatus may be used to address complex or multi-stage problems, situations, and/or questions. For example, a first apparatus may be used to predict tumor development, whereas a second apparatus may predict the outcome of tumor treatment such as surgery and/or radiotherapy. In certain embodiments, the output (e.g., prediction) of the first apparatus may serve as input to the second apparatus. In certain embodiments, the first apparatus and the second apparatus may share some or all of their inputs or internal states. In certain embodiments, the first apparatus and the second apparatus may be trained so that the accuracy of predictions and/or other metrics of one or both apparatuses is optimized, for example by adversarial training techniques.

Although embodiments of the present disclosure are directed to techniques used to aid in diagnostics and treatment of cancer, in certain other embodiments, the techniques described herein may be used to aid in diagnostics and treatment of other medical and/or other veterinary conditions, for example, by replacing computation model 102 of tumor growth with a computation model of other developmental and/or physiological processes, either normal or pathological. An example of such application may be application in sports, in battlefield, or with respect to other injury or trauma. Both the mechanism of the trauma itself and the body response to the trauma and/or treatment may be accounted for by the computational model(s) and/or the machine learning model(s) as described herein. For example, such models may be used to assist in personnel training, triage, treatment, follow-up, rehabilitation, and/or the like. Such models may also be used in the development and/or evaluation of better protective equipment, prosthetics, battlefield tactics, sports, medical procedures (e.g. concussion protocol), and/or the like.

### Example Clauses

Implementation details of various aspects of the present disclosure are described in the following numbered clauses.

Clause 1: A method of training a tumor detection model, the method comprising: processing an input scan image of a tumor in a first state using a first computational model configured to simulate growth of the tumor in a first configuration to generate model output images of the simulated tumor comprising at least a first model output image representing the tumor in a second state, wherein the first model output image comprises a timestamp associated with the second state; training a first machine learning model to convert the first model output image to a first synthesized scan image; and training a second machine learning model to detect the simulated tumor in image data using the first synthesized scan image and the input scan image.

Clause 2: The method of Clause 1, wherein the first computational model is configured to simulate the growth of the tumor when a treatment is applied or without the treatment applied.

Clause 3: The method of any one of Clauses 1-2, further comprising: processing the input scan image of the tumor in the first state using a second computational model configured to simulate tissue movement around the tumor in the first configuration, wherein the first machine learning model is trained to generate the first synthesized scan image further based on the tissue movement simulated by the second computational model.

Clause 4: The method of any one of Clauses 1-3, further comprising: training a third machine learning model to generate a predicted model output image of the simulated tumor at a predetermined time step for a period of time, wherein a plurality of predicted model output images of the simulated tumor are generated and represent different states of the tumor for the period of time; training the first machine learning model to convert the plurality of predicted model output images to a plurality of synthesized scan images; and training the second machine learning model to at least one of: detect the simulated tumor in each of the plurality of synthesized scan images; or generate a second synthesized scan image representing the tumor in a state in time between at least two of the plurality of predicted model output images.

Clause 5: The method of Clause 4, wherein the at least one of the first machine learning model, the second machine learning model, or the third machine learning model takes part in at least one of continuous learning or federated learning after deployment.

Clause 6: The method of any one of Clauses 4-5, wherein the plurality of predicted model output images of the simulated tumor represent the different states of the tumor when a treatment is applied or without the treatment applied.

Clause 7: The method of any one of Clauses 1-6, wherein the first model output image representing the tumor in the second state comprises at least one annotated margin of the simulated tumor.

Clause 8: The method of Clause 7, wherein the at least one annotated margin of the simulated tumor is generated by the first computational model, generated by a separate model, or manually added to the first model output image.

Clause 9: The method of any one of Clauses 1-8, wherein the first synthesized scan image comprises at least one annotated margin of the simulated tumor.

Clause 10: The method of Clause 9, wherein the at least one annotated margin of the simulated tumor is generated by the first machine learning model, generated by a separate model, or manually added to the first synthesized scan image.

Clause 11: The method of any one of Clauses 1-10, wherein the first synthesized scan image comprises a two-dimensional (2D) representation or a three-dimensional (3D) representation of the second state of the tumor.

Clause 12: The method of Clause 11, wherein the 2D representation or the 3D representation comprise at least one of: a computerized tomography (CT) scan, a magnetic resonance imaging (Mill) image, a functional MRI (fMRI) image, a positron emission tomography (PET) scan, or a 3D model.

Clause 13: The method of any one of Clauses 1-12, further comprising: processing the input scan image of the tumor using the first computational model in a second configuration to generate a second model output image representing the tumor in a third state; training the first machine learning model to convert the second model output image to a second synthesized scan image; and training the second machine learning model to detect the simulated tumor in image data using the second synthesized scan image and the input scan image.

Clause 14: The method of any one of Clauses 1-13, wherein the first machine learning model comprises a generative adversarial network (GAN).

Clause 15: The method of any one of Clauses 1-14, wherein the first machine learning model comprises a diffusion network.

Clause 16: The method of any one of Clauses 1-15, wherein the second machine learning model comprises a predictor neural network.

Clause 17: The method of any one of Clauses 1-16, wherein the second machine learning model comprises an autoencoder.

Clause 18: The method of any one of Clauses 1-17, wherein the second machine learning model comprises an encoder-decoder or a future encoder.

Clause 19: A method of generating synthesized scan images representing a tumor, the method comprising: processing an input scan image of the tumor in a first state using a first computational model configured to simulate growth of the tumor in a first configuration to generate model output images of the simulated tumor comprising at least a first model output image representing the tumor in a second state, wherein the first model output image comprises a timestamp associated with the second state; converting the first model output image to a first synthesized scan image using a first machine learning model trained to convert model output images to synthesized scans; and detecting the simulated tumor in the first synthesized scan image using a second machine learning model trained to simulated tumors in image data.

Clause 20: A method of generating synthesized scan images representing a tumor, the method comprising: generating predicted model output images of a tumor simulation for the tumor at a predetermined time step for a period of time, wherein a plurality of predicted model output images of the simulated tumor are generated and represent different states of the tumor for the period of time; converting the plurality of predicted model output images to a plurality of synthesized scan images using a first machine learning model trained to convert predicted model output images to synthesized scans; and performing at least one of: detecting the simulated tumor in each of the plurality of synthesized scan images using a second machine learning model trained to simulated tumors in image data; or generating one or more synthesized scan images representing the tumor in a state in time between at least two of the plurality of predicted model output images.

Clause 21: A processing system, comprising: a memory having executable instructions stored thereon; and a processor configured to execute the executable instructions to cause the processing system to perform the operations of any one of Clauses 1 through 20.

Clause 22: A processing system, comprising: means for performing the operations of any one of Clauses 1 through 20.

Clause 23: A computer-readable medium having executable instructions stored thereon which, when executed by a processor, causes the processor to perform the operations of any one of clauses 1 through 20.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. §112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method of training a tumor detection model, the method comprising:
processing an input scan image of a tumor in a first state using a first computational model configured to simulate growth of the tumor in a first configuration to generate model output images of the simulated tumor comprising at least a first model output image representing the tumor in a second state, wherein the first model output image comprises a timestamp associated with the second state;
training a first machine learning model to convert the first model output image to a first synthesized scan image; and
training a second machine learning model to detect the simulated tumor in image data using the first synthesized scan image and the input scan image.

2. The method of claim 1, wherein the first computational model is configured to simulate the growth of the tumor when a treatment is applied or without the treatment applied.

3. The method of claim 1, further comprising:
processing the input scan image of the tumor in the first state using a second computational model configured to simulate tissue movement around the tumor in the first configuration,
wherein the first machine learning model is trained to generate the first synthesized scan image further based on the tissue movement simulated by the second computational model.

4. The method of claim 1, further comprising:
training a third machine learning model to generate a predicted model output image of the simulated tumor at a predetermined time step for a period of time, wherein a plurality of predicted model output images of the simulated tumor are generated and represent different states of the tumor for the period of time;
training the first machine learning model to convert the plurality of predicted model output images to a plurality of synthesized scan images; and
training the second machine learning model to at least one of:
detect the simulated tumor in each of the plurality of synthesized scan images; or
generate a second synthesized scan image representing the tumor in a state in time between at least two of the plurality of predicted model output images.

5. The method of claim 4, wherein the at least one of the first machine learning model, the second machine learning model, or the third machine learning model takes part in at least one of continuous learning or federated learning after deployment.

6. The method of claim 4, wherein the plurality of predicted model output images of the simulated tumor represent the different states of the tumor when a treatment is applied or without the treatment applied.

7. The method of claim 1, wherein the first model output image representing the tumor in the second state comprises at least one annotated margin of the simulated tumor.

8. The method of claim 7, wherein the at least one annotated margin of the simulated tumor is generated by the first computational model, generated by a separate model, or manually added to the first model output image.

9. The method of claim 1, wherein the first synthesized scan image comprises at least one annotated margin of the simulated tumor.

10. The method of claim 9, wherein the at least one annotated margin of the simulated tumor is generated by the first machine learning model, generated by a separate model, or manually added to the first synthesized scan image.

11. The method of claim 1, wherein the first synthesized scan image comprises a two-dimensional (2D) representation or a three-dimensional (3D) representation of the second state of the tumor.

12. The method of claim 11, wherein the 2D representation or the 3D representation comprise at least one of:
a computerized tomography (CT) scan,
a magnetic resonance imaging (Mill) image,
a functional MRI (fMRI) image,
a positron emission tomography (PET) scan, or
a 3D model.

13. The method of claim 1, further comprising:
processing the input scan image of the tumor using the first computational model in a second configuration to generate a second model output image representing the tumor in a third state;
training the first machine learning model to convert the second model output image to a second synthesized scan image; and
training the second machine learning model to detect the simulated tumor in image data using the second synthesized scan image and the input scan image.

14. The method of claim 1, wherein the first machine learning model comprises a generative adversarial network (GAN).

15. The method of claim 1, wherein the first machine learning model comprises a diffusion network.

16. The method of claim 1, wherein the second machine learning model comprises a predictor neural network.

17. The method of claim 1, wherein the second machine learning model comprises an autoencoder.

18. The method of claim 1, wherein the second machine learning model comprises an encoder-decoder or a future encoder.

19. A method of generating synthesized scan images representing a tumor, the method comprising:
processing an input scan image of the tumor in a first state using a first computational model configured to simulate growth of the tumor in a first configuration to generate model output images of the simulated tumor comprising at least a first model output image representing the tumor in a second state, wherein the first model output image comprises a timestamp associated with the second state;
converting the first model output image to a first synthesized scan image using a first machine learning model trained to convert model output images to synthesized scans; and
detecting the simulated tumor in the first synthesized scan image using a second machine learning model trained to simulated tumors in image data.

20. A method of generating synthesized scan images representing a tumor, the method comprising:
generating predicted model output images of a tumor simulation for the tumor at a predetermined time step for a period of time, wherein a plurality of predicted model output images of the simulated tumor are generated and represent different states of the tumor for the period of time;
converting the plurality of predicted model output images to a plurality of synthesized scan images using a first machine learning model trained to convert predicted model output images to synthesized scans; and
performing at least one of:
detecting the simulated tumor in each of the plurality of synthesized scan images using a second machine learning model trained to simulated tumors in image data; or
generating one or more synthesized scan images representing the tumor in a state in time between at least two of the plurality of predicted model output images.
